(19) United States
(12) Patent Application Publication
(10) Pub. No.: US 2026/0234810 A1
(43) Pub. Date: Aug. 13, 2026

(54) PRE-TREATMENT FOR SAM REMOVAL IMPROVEMENT WITH LESS CARBON RESIDUE

(71) Applicant: Applied Materials, Inc., Santa Clara, CA (US)

(72) Inventors: Zheng JU, Santa Clara, CA (US); Jiajie CEN, San Jose, CA (US); Ruinan ZHOU, Santa Clara, CA (US); Zhiyuan WU, San Jose, CA (US); Kevin KASHEFI, Dublin, CA (US)

(21) Appl. No.: 19/233,035

(22) Filed: Jun. 10, 2025

Related U.S. Application Data
(60) Provisional application No. 63/757,287, filed on Feb. 11, 2025.

(51) Int. Cl. C23C 30/00

(52) U.S. Cl. CPC C23C 30/005

(57) ABSTRACT

A method of forming a microelectronic device includes pre-treating a metal surface formed on a substrate, depositing a self-assembled monolayer (SAM) selectively on the metal surface against a dielectric surface formed on the substrate, depositing a barrier layer selectively on the dielectric surface against the SAM, and removing the SAM.

### CROSS-REFERENCE TO RELATED APPLICATIONS

This application claims benefit of U.S. provisional patent application Ser. No. 63/757,287, filed Feb. 11, 2025, which is herein incorporated by reference.

### BACKGROUND

### Field

Embodiments of the present invention generally relate to methods of forming interconnect structures in microelectronic devices. More particularly, embodiments of the disclosure are directed to methods of reducing carbon residue after removal of self-assembled monolayers selectively deposited on metal surfaces against dielectric surfaces during formation of interconnect structures in microelectronic devices.

### Description of the Related Art

The semiconductor industry faces many challenges in the pursuit of device miniaturization which involves rapid scaling of nanoscale features. Such issues include the introduction of complex fabrication steps such as multiple lithography steps and integration of high-performance materials. Selective deposition has shown promise in device miniaturization as it has the potential to remove costly lithographic steps by simplifying integration schemes.

Selective deposition of materials can be accomplished in a variety of ways. A chemical precursor may react selectively with one surface relative to another surface (metallic or dielectric). Process parameters such as pressure, substrate temperature, precursor partial pressures, and/or gas flows might be modulated to modulate the chemical kinetics of a particular surface reaction. Another possible scheme involves surface pretreatments that can be used to activate or deactivate a surface of interest to an incoming film deposition precursor. For example, a self-assembled monolayer (SAM) can be formed on a surface to prevent subsequent deposition on that surface.

The issues with current state of the SAM include some carbon residue after SAM removal. This carbon residue is more critical now to be resolved, as it may cause a higher overall effective resistance Rc of an electrical circuit with carbon impurity and Rc drift issue. Carbon residue on metals and other surface may also cause time dependent dielectric breakdown (TDDB) failures.

Accordingly, there is an ongoing need in the art for methods of reducing carbon residue after removing self-assembled monolayers on metal surfaces during formation of interconnect structures in microelectronic devices.

### SUMMARY

Embodiments of the present disclosure provide a method of forming a microelectronic device. The method includes pre-treating a metal surface formed on a substrate, depositing a self-assembled monolayer (SAM) selectively on the metal surface against a dielectric surface formed on the substrate, depositing a barrier layer selectively on the dielectric surface against the SAM, and removing the SAM.

Embodiments of the present disclosure also provide a method of forming a microelectronic device. The method includes pre-cleaning and pre-treating a top surface of a first metal layer exposed within a feature opening extending through a dielectric layer that is formed on the first metal layer, depositing a blocking layer selectively on the top surface of the first metal layer exposed within the feature opening against sidewalls of the feature opening, depositing a barrier layer selectively on the sidewalls of the feature opening against the blocking layer, removing the blocking layer, and filling the feature opening with a second metal layer.

Embodiments of the present disclosure further provide a method of removal of self-assembled monolayer (SAM) from a metal surface. The method includes soaking the metal surface in ammonia (NH₃) gas supplied at a flow rate of between 500 sccm and 3000 sccm, for a time duration of between 10 seconds and 60 seconds, subsequent to soaking the metal surface, depositing a self-assembled monolayer (SAM) on the metal surface, and removing the SAM from the metal surface, wherein carbon (C) residue on the metal surface after the removing the SAM is reduced as compared to a SAM removal without the soaking the metal surface.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates a process flow diagram of a method of forming a microelectronic device in accordance with one or more embodiment of the disclosure.

FIG. 2 illustrates a cross-sectional schematic view of a microelectronic device prior to a selective deposition process in accordance with one or more embodiments of the disclosure.

FIG. 3 illustrates a cross-sectional schematic view of the microelectronic device of FIG. 2 after formation of a blocking layer in accordance with one or more embodiments of the disclosure.

FIG. 4 illustrates a cross-sectional schematic view of the microelectronic device of FIG. 3 after formation of a barrier layer in accordance with one or more embodiments of the disclosure.

FIG. 5 illustrates a cross-sectional schematic view of the microelectronic device of FIG. 4 after removal of the blocking layer in accordance with one or more embodiments of the disclosure.

FIG. 6 illustrates a cross-sectional schematic view of the microelectronic device of FIG. 5 after formation of a metal liner in accordance with one or more embodiments of the disclosure.

FIG. 7 illustrates a cross-sectional schematic view of the microelectronic device of FIG. 6 after formation of a second metal layer in accordance with one or more embodiments of the disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments described herein are directed to methods of reducing carbon residue after removing self-assembled monolayers selectively deposited on metal surfaces against dielectric surfaces during formation of interconnect structures in microelectronic devices. The methods described herein include applying a pre-treatment process after metal preclean process, prior to an SAM soak process to mitigate and weaken the bonding between a self-assembled monolayer (SAM) and the metal surfaces. The pre-treatment has minimal impact on selectivity in deposition of a barrier layer between the SAM and the dielectric surfaces and reduces carbon residue after SAM removal process with CCP plasma.

The inventors have shown that a pre-treatment process improves the removal efficiency of a SAM from a metal surface as compared to an SAM removal without the pre-treatment process. Carbon (C) residue on the metal surface after an SAM removal is reduced by between about 20 % and about 50 %, for example, about 50% due to the pre-treatment process.]

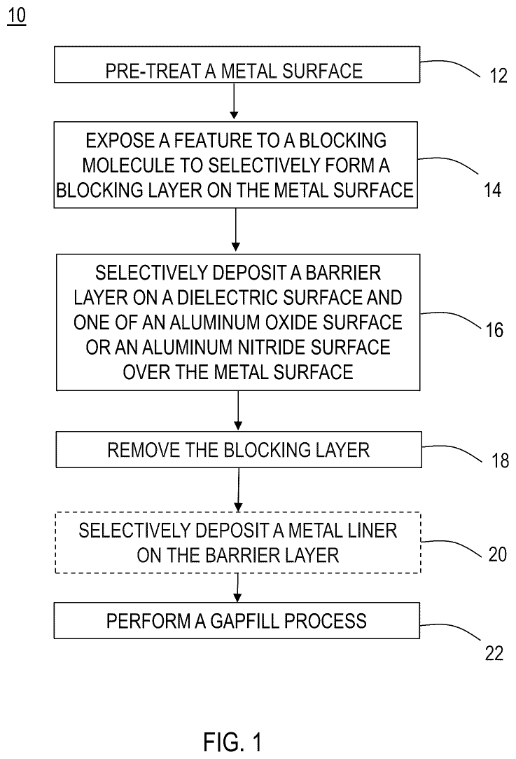

A process flow of a method 10 of forming a microelectronic device according to one or more embodiments of the disclosure is described with respect to FIGS. 1 to 7. The method 10 described herein can be used to form semiconductor devices or interconnects for semiconductor devices.

The method 10 in FIG. 1 includes pre-cleaning and pre-treating a metal surface of a feature extending into a semiconductor substrate (operation 12); exposing the feature to blocking molecule, to form a blocking layer on the metal surface, the feature defining a gap including the metal surface, a dielectric surface, and an aluminum oxide surface or an aluminum nitride surface, the blocking layer forming selectively on the metal surface against the dielectric surface and one of an aluminum oxide surface or an aluminum nitride surface (operation 14); selectively depositing a barrier layer on the dielectric surface and one of the aluminum oxide surface or the aluminum nitride surface against the blocking layer (operation 16); removing the blocking layer (operation 18); optionally, selectively depositing a metal liner on the barrier layer (operation 20); and performing a gapfill process (operation 22).

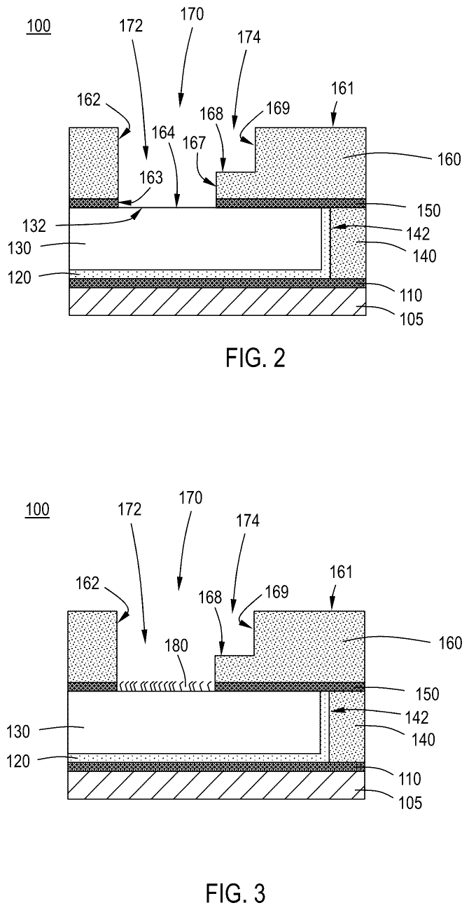

FIG. 2 illustrates an exemplary embodiment of an electronic device 100. The electronic device 100 includes a substrate 105 with a first layer 110 formed thereon. The substrate 105 can be any suitable substrate material for use with an electronic device. In some embodiments, the substrate 105 is a semiconductor substrate. In some embodiments, the substrate 105 can include additional layers of dielectrics, metals, etch stop layers and semiconductor layers including underlying circuits (e.g., transistors, capacitors) that have been formed in prior processes.

The first layer 110 is an optional layer. In some embodiments, the first layer 110 is omitted. In some embodiments, the first layer 110 is formed of a lamination of more than one layer. The first layer 110 can have any suitable function including, but not limited to, acting as an etch stop layer for previous or future microelectronic device manufacturing operations.

A first dielectric layer 140 is formed on a portion of the first layer 110. The first dielectric layer 140 has an inner sidewall 142 which defines a boundary of the first dielectric layer 140, leaving an opening. The opening can be, for example, a trench for a first metallization layer of the electronic device 100.

The first dielectric layer 140 can be any suitable material formed by any suitable technique. In some embodiments, the first dielectric layer 140 is formed of an oxide, a nitride, or any combination thereof. In some embodiments, the first dielectric layer 140 is formed of silicon oxide (SiO₂). The first dielectric layer 140 may be deposited by one or more of chemical vapor deposition (CVD), atomic layer deposition (ALD), plasma-enhanced chemical vapor deposition (PECVD), plasma-enhanced atomic layer deposition (PEALD), physical vapor deposition (PVD), or spin-on techniques.

The dielectric surface of the first dielectric layer 140 on the substrate 105 may be formed of any suitable dielectric materials. Suitable dielectric materials include, but are not limited to, oxides (e.g., silicon oxides (SiO₂)), low-κ dielectric materials, and high-k dielectric materials. In some embodiments, the first dielectric layer 140 is formed of silicon oxide (SiO₂), silicon nitride (Si₃N₄), silicon carbooxynitride (SiCON), silicon oxycarbide (SiOC), aluminum oxide (AlOₓ), aluminum nitride (AlNₓ), or any combination thereof.

In some embodiments, a liner 120 is formed on the top surface of the first layer 110 and abutting the inner sidewall 142 of the first dielectric layer 140. The liner 120 acts as one or more of an adhesion layer, barrier layer, or liner. The liner 120 can be any suitable material, including, but not limited to, oxides and nitrides. The liner 120 can be formed by any suitable technique. In some embodiments, the liner 120 is formed as a conformal film by atomic layer deposition (ALD).

In some embodiments, a first metal layer 130 is formed on the liner 120 and forms a first metallization layer. The first metal layer 130 can be any suitable material deposited by any suitable technique. Suitable metal materials include, but are not limited to, metals, metal nitrides, metal alloys, and other conductive materials. In some embodiments, the first metal layer 130 is formed of copper (Cu), cobalt (Co), tungsten (W), molybdenum (Mo), ruthenium (Ru), titanium nitride (TiN), or any combination thereof.

In some embodiments, an etch stop layer 150 is formed on the first metal layer 130. The etch stop layer 150 can be any suitable material formed by any suitable technique.

In some embodiments, the etch stop layer 150 is formed of aluminum oxide (AlOₓ), such as Al₂O₃, aluminum nitride (AlNₓ), or combination thereof.

In some embodiments, a second dielectric layer 160 is formed on the etch stop layer 150. The second dielectric layer 160 can be any suitable material formed by any suitable technique. In some embodiments, the second dielectric layer 160 is formed of an oxide, a nitride, or combination thereof. In some embodiments, the second dielectric layer 160 is formed of a low-κ dielectric material. In some embodiments, the second dielectric layer 160 is formed of silicon oxide. The second dielectric layer 160 may be deposited by one or more of chemical vapor deposition (CVD), atomic layer deposition (ALD), plasma-enhanced chemical vapor deposition (PECVD), plasma-enhanced atomic layer deposition (PEALD), physical vapor deposition (PVD), or spin-on techniques.

The dielectric surface of the second dielectric layer 160 may be formed of any suitable dielectric materials. Suitable dielectric materials include, but are not limited to, oxides (e.g., silicon oxides), low-κ dielectric materials, and high-k dielectrics. In some embodiments, the second dielectric layer 160 is formed of silicon oxide (SiO₂), silicon nitride (Si₃N₄), silicon carbooxynitride (SiCON), silicon oxycarbide (SiOC), aluminum oxide (AlOₓ), aluminum nitride (AlNₓ), or any combination thereof. In some embodiments, the second dielectric layer 160 is formed of the same material as the first dielectric layer 140. In some embodiments, the second dielectric layer 160 is formed of a different material than the first dielectric layer 140.

The substrate 105 including the second dielectric layer 160, the etch stop layer 150 and the first metal layer 130 has a feature opening 170 formed therein. The feature opening 170 is formed in the second dielectric layer 160 and the etch stop layer 150 exposing a top surface 132 of the first metal layer 130. The feature opening 170 illustrated has a via portion 172 and a trench portion 174.

The via portion 172 extends through the second dielectric layer 160 and the etch stop layer 150 to the first metal layer 130. The via portion 172 exposes the top surface 132 of the first metal layer 130. The top surface 132 of the first metal layer 130 forms a bottom surface 164 of the via portion 172. The via portion 172 is bounded on one side by a sidewall 162 of the second dielectric layer 160 and a sidewall 163 of the etch stop layer 150. The via portion 172 is bounded on another side by a lower sidewall 167 of the second dielectric layer 160 and the sidewall 163 of the etch stop layer 150. It should be noted that the via portion 172 may have a circular cross-section and that the use of a first side and a second side (e.g., one side and another side) to describe the via portion 172 is for descriptive purposes based on the cross-sectional views in the Figures. When the via portion 172 is a cylindrical hole, the sidewalls of the dielectric layer and the etch stop layer are continuous so that there is effectively a single sidewall, rather than a first side and second side (e.g., one side and another side) that appears in the cross-section.

The trench portion 174 has a bottom surface 168 formed from the second dielectric layer 160 and is bounded on one side by an upper sidewall 169 which is formed of the second dielectric layer 160. The trench portion 174 has an open side where the via portion 172 passes through the second dielectric layer 160 and etch stop layer 150.

The portion of the feature opening 170 not bounded by the via portion 172 and the trench portion 174 can be collectively referred to as a gap.

The substrate 105 includes a metal surface (the bottom surface 164 of the via portion 172), a dielectric surface (the sidewall 162, the lower sidewall 167, the bottom surface 168, the upper sidewall 169, and a top surface 161) and one or more of an aluminum oxide surface or an aluminum nitride surface (the sidewall 163 of etch stop layer 150).

The metal surface (the bottom surface 164 of the via portion 172) is pre-cleaned and pre-treated to mitigate the bonding between a blocking layer 180 to be formed on the metal surface and the metal surface (operation 16). The pre-treatment of the metal surface includes soaking the substrate 105 in nitrogen containing solution or gas, such as ammonia (NH₃). In some embodiments, the nitrogen containing gas is supplied at a flow rate of between about 500 sccm and about 3000 sccm, for a time duration of between about 10 seconds and about 60 seconds, for example, about 40 seconds.

FIG. 3 illustrates the electronic device 100 of FIG. 2 after selectively depositing a blocking layer 180 (operation 14). The substrate 105 including the metal surface (the bottom surface 164 of the via portion 172), the dielectric surface (the sidewall 162, the lower sidewall 167, the bottom surface 168, the upper sidewall 169, and the top surface 161), and one or more of the aluminum oxide surface or the aluminum nitride surface (the sidewall 163 of the etch stop layer 150) is exposed to a blocking molecule,, to form the blocking layer 180. The blocking layer 180 is selectively formed on the metal surface (the bottom surface 164 of the via portion 172) against the dielectric surface (the sidewall 162, the lower sidewall 167, the bottom surface 168, the upper sidewall 169, and the top surface 161) and the aluminum oxide surface or the aluminum nitride surface (the sidewall 163 of the etch stop layer 150).

In some embodiments, the blocking molecule used to deposit the blocking layer 180 is formed of alkyne molecules. In some embodiments, the blocking molecule is reacted with a metal surface (including but not limited to copper (Cu), cobalt (Co), tungsten (W), molybdenum (Mo), ruthenium (Ru), or titanium nitride (TiN)) in solution or vapor phase. In some embodiments, the metal surface is cleaned prior to reaction with the blocking molecules.

The dielectric surface of the substrate 105 may be formed of any suitable dielectric materials. Suitable dielectric materials include, but are not limited to, oxides (e.g., silicon oxides), low-k dielectric materials, and high-k dielectric materials.

The metal surface of the substrate may be formed of any suitable metal materials. Suitable metal materials include, but are not limited to, metals, metal nitrides, metal alloys, and other conductive materials. In some embodiments, the metal surface is formed of copper (Cu), cobalt (Co), tungsten (W), molybdenum (Mo), ruthenium (Ru), or titanium nitride (TiN), or any combination thereof.

In some embodiments, forming the blocking layer 180 in operation 14 includes soaking the substrate 105 in the blocking molecule. In some embodiments, forming the blocking layer 180 includes exposing the substrate 105 to pulses of the blocking chemistry. The pulses of blocking chemistry can be any suitable duration and occur any suitable number of times. In some embodiments, during formation of the blocking layer 180 occurs with greater than 1, 10, 100, 250, 500 or 1000 pulses of blocking layer chemistry. In some embodiments, the total time for exposure to the blocking chemistry is greater than 1 second, 10 seconds, 100 seconds, 500 seconds or 1000 seconds.

In some embodiments, the Alkyne groups cross-link with each other after deposition. In some embodiments, the blocking layer contains substantially no crosslinking between the Alkyne groups.

In some embodiments, the substrate 105 is cleaned prior to exposing the substrate 105 to the blocking molecule. In some embodiments, only the metal surface of the substrate 105 is cleaned prior to exposing the substrate to the blocking molecule. In some embodiments, the substrate 105 or the metal surface of the substrate 105 is cleaned with a hydrogen plasma. In some embodiments, the hydrogen plasma is a conductively coupled plasma (CCP). In some embodiments, the hydrogen plasma is an inductively coupled plasma (ICP). In some embodiments, the hydrogen plasma is formed of plasma of H₂.

The blocking layer 180 is formed at a temperature that is favorable to close packing of the self-assembled monolayer of blocking chemistry species. In some embodiments, the substrate 105 is maintained at a temperature in the range of 100° C. to 500° C., or in the range of 150° C. to 450° C., or in the range of 200° C. to 400° C., or in the range of 225° C. to 350° C., or in the range of 250° C. to 350° C., or in the range of 250° C. to 300° C.

Pre-cleaning of the substrate can occur at any suitable temperature depending on, for example, the cleaning technique. In some embodiments, pre-cleaning of the substrate occurs at a temperature in the range of 200° C. to 500° C., or in the range of 300° C. to 400° C.

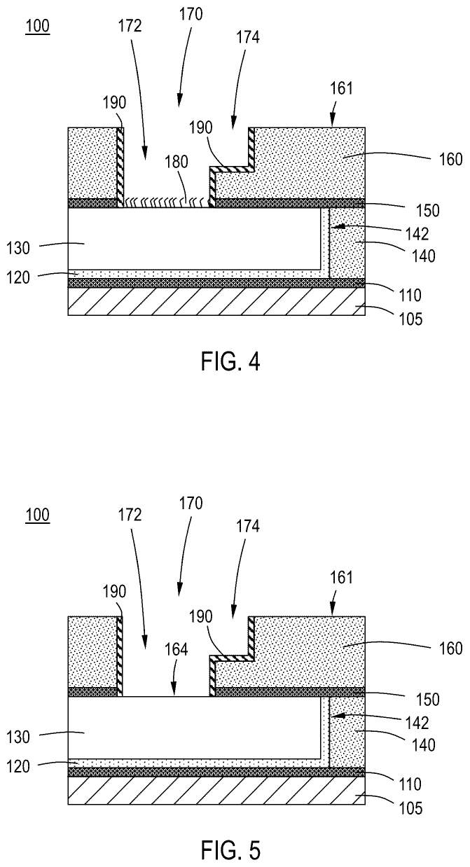

FIG. 4 illustrates the electronic device 100 of FIG. 3 after formation of a barrier layer 190 on the surfaces of the second dielectric layer 160 and the etch stop layer 150 (operation 16).

In some embodiments, the barrier layer 190 is a dielectric layer that is selectively deposited on the dielectric surface after deposition of the blocking layer 180. In some embodiments, the barrier layer 190 is formed of silicon nitride (Si₃N₄). Deposition of silicon nitride (Si₃N₄) can be performed through any suitable process. In one or more embodiments, the barrier layer 190 is formed by an atomic layer deposition (ALD) process. Suitable processes may include exposure of the substrate to a silicon halide and ammonia. Suitable silicon halides include, but are not limited to dichlorosilane (DCS), trichlorosilane (TCS), tetrachlorosilane (SiCl₄), tetrabromosilane (SiBr₄), tetraiodosilane (SiI₄), and hexachlorodisilane (HCDS).

In some embodiments, the barrier layer 190 is formed of tantalum nitride (TaN). In some embodiments, the tantalum nitride (TaN) is deposited by a thermal atomic layer deposition (ALD) process. As used in this manner, a thermal process does not include a plasma. In some embodiments, the tantalum nitride is deposited using pentakis(dimethylamino)tantalum (PDMAT) and ammonia in a thermal ALD process.

In some embodiments, exposing the substrate to the blocking molecule (operation 14) is repeated after deposition of the barrier layer 190 (operation 16) to regenerate the blocking layer 180. In some embodiments, the barrier layer 190 is deposited again after the blocking layer 180 is regenerated. In some embodiments, exposure of the substrate 105 to a blocking molecule and depositing the barrier layer 190 is repeated until the barrier layer 190 has reached a predetermined thickness.

The exposure to the surface blocking chemistry, or the regeneration of the blocking layer 109, can be performed once or repeated after a number of deposition cycles or after a predetermined film thickness is formed. In some embodiments, the barrier layer 190 is deposited with a thickness in the range of about 5 Å to about 50 Å, or in the range of about 10 Å to about 40 Å, or in the range of about 15 Å to about 35 Å before the blocking layer 180 is regenerated.

In some embodiments, the first metal layer 130 is formed of tungsten (W), the etch stop layer 150 is formed of aluminum oxide, aluminum nitride, or combination thereof, the second dielectric layer 160 is formed of silicon oxide (SiO₂), and the barrier layer 190 is formed of tantalum nitride (TaN).

In some embodiments, the metal surface (the first metal layer 130 and the bottom surface 164) is formed of tungsten (W). In some embodiments, the metal surface (the first metal layer 130 and the bottom surface 164) comprises cobalt *Co).

In some embodiments, the barrier layer 190 is selectively deposited on the top surface 161, the sidewall 162, the lower sidewall 167, the bottom surface 168, the upper sidewall 169 of the second dielectric layer 160 in the feature opening 170 and the surface (the sidewall 163) of the etch stop layer 150 after formation of the blocking layer 180 on the underlying metal surface (the bottom surface 164 of the via portion 172). Stated differently, the barrier layer 190 is deposited on the sidewall 162, the sidewall 163 of the via portion 172 and the bottom surface 168 and the upper sidewall 169 of the trench portion 174 of the feature opening 170. In some embodiments, the barrier layer 190 is formed on the sidewalls to the blocking layer 180 on the metal surface. In some embodiments, a small gap is formed between the bottom surface 164 of the via portion 172 and the bottom edge of the barrier layer 190 due to the presence of the blocking layer 180.

Deposition of tantalum nitride (TaN) can be performed by any suitable process. In some embodiments, the barrier layer 190 is formed of one or more of tantalum nitride (TaN), titanium nitride (TiN), silicon nitride (Si₃N₄), titanium silicon nitride (TiSiN), tantalum silicon nitride (TaSiN), and silicon oxynitride (SiON).

In the embodiment illustrated in the Figures, the barrier layer 190 is formed on the sidewall 162 of the via portion 172, the lower sidewall 167, the bottom surface 168 and the upper sidewall 169 of the trench portion 174 of the feature opening 170. The Figures do not show the barrier layer 190 material on the top surface 161 of the second dielectric layer 160. In some embodiments, deposition of the barrier layer 190 results in formation of the barrier layer 190 on the top surface 161 of the second dielectric layer 160. The substrate 105 is then subjected to a process to remove the barrier layer 190 from the top surface 161 of the second dielectric layer 160, for example, by chemical mechanical planarization (CMP).

FIG. 5 illustrates the electronic device 100 of FIG. 4 after removal of the blocking layer 180 to expose the bottom surface 164 of the via portion 172, which is the top surface of the first metal layer 130 (operation 18).

The blocking layer 180 can be removed by any suitable technique (operation 18). In some embodiments, removing the blocking layer 180 includes exposing the blocking layer 180 to hydrogen (H₂) plasma. In some embodiments, the plasma is a capacitively coupled plasma (CCP). In some embodiments, the plasma is an inductively coupled plasma (ICP).

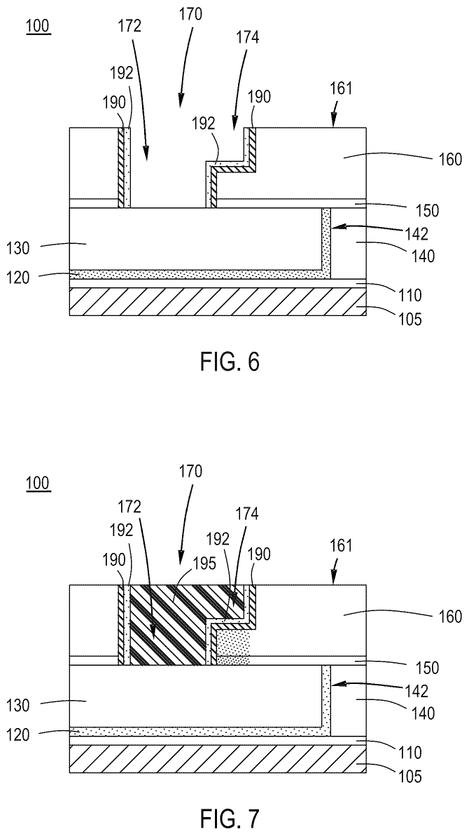

FIG. 6 illustrates the electronic device of FIG. 5 after deposition of a metal liner 192 on the barrier layer 190 (optional operation 20). In some embodiments, the metal liner 192 is selectively deposited on the barrier layer 190 by any suitable deposition process described herein. In some embodiments, the metal liner 192 is formed of ruthenium (Ru), cobalt (Co), molybdenum (Mo), tantalum (Ta), or any combination thereof. In some embodiments, the metal liner 192 is formed to a thickness in the range of 5 Å to 25 Å.

In some embodiments, the metal liner 192 is formed of cobalt (Co), the barrier layer 190 is formed of tantalum nitride (TaN), the etch stop layer 150 is formed of aluminum oxide aluminum oxide, the second dielectric layer 160 is formed of silicon oxide, and the first metal layer 130 is formed of tungsten.

FIG. 7 illustrates the electronic device of FIG. 6 after the gapfill process (operation 22) to deposit a second metal layer 195 in the feature opening 170. In some embodiments, the second metal layer 195 is deposited to fill the gap, i.e., the portion of the feature opening 170 not bounded by the via portion 172 and the trench portion 174. The second metal layer 195 is formed on the top surface of the first metal layer 130 (the bottom surface 164 of the via portion 172). In some embodiments, the second metal layer 195 is formed on the top surface of the metal liner 192. In some embodiments, the second metal layer 195 is formed of copper (Cu) or cobalt (Co). In some embodiments, the second metal layer 195 is formed of copper (Cu). In some embodiments, the second metal layer 195 is formed of cobalt (Co).

In some embodiments, the second metal layer 195 is formed of copper (Cu), the metal liner 192 is formed of cobalt (Co), the barrier layer 190 is formed of tantalum nitride (TaN), the etch stop layer 150 is formed of aluminum oxide (Al₂O₃), the second dielectric layer 160 is formed of silicon oxide, the first metal layer 130 is formed of tungsten (W), and the second metal layer 195 is formed on the top surface of the first metal layer 130 that is exposed through the via portion 172 of the feature opening 170 and on the metal liner 192.

In embodiments where the metal liner 192 is not present, the second metal layer 195 is formed on the top surface of the first metal layer 130 that is exposed through the via portion 172 of the feature opening 170, the barrier layer 190 on the sidewall 162 of the via portion 172, and on the barrier layer 190 of the lower sidewall 167, the bottom surface 168 and the upper sidewall 169 of the trench portion 174.

In some embodiments, a blanket deposition process deposits the second metal layer 195 into the feature opening 170 and on the top surface 161 of the second dielectric layer 160. The second metal layer 195 is formed on the top surface 161 of the second dielectric layer 160 can be removed by any suitable technique including, but not limited to, etching and chemical mechanical planarization.

One or more embodiments of the disclosure are directed to a non-transitory computer readable medium including instructions, that, when executed by a controller of a processing chamber, cause the processing chamber to perform the operations of the method 10. In some embodiments, the non-transitory computer readable medium includes instructions, that, when executed by a controller of a processing chamber, cause the processing chamber to: pre-treat a metal surface of a feature extending into a semiconductor substrate (operation 12); expose the feature to blocking molecule to form a blocking layer, the feature defining a gap including the metal surface, a dielectric surface, and an aluminum oxide surface or an aluminum nitride surface, the blocking layer forming selectively on the metal surface against the dielectric surface and one of the aluminum oxide surface or the aluminum nitride surface (operation 14); selectively deposit a barrier layer on the dielectric surface and one of the aluminum oxide surface or the aluminum nitride surface against the blocking layer (operation 16); remove the blocking layer (operation 18); optionally, selectively deposit a metal liner on the barrier layer (operation 20); and performing a gapfill process (operation 22).

In the embodiments described herein, methods of reducing carbon residue after removing self-assembled monolayers deposited on metal surfaces against dielectric surfaces during formation of interconnect structures in microelectronic devices. This pre-treatment allows much less carbon residue on a metal surface, leading to more Rc gain and less Rc drift issue.

Advantages of the method described herein are summarized in the Appendix.

While the foregoing is directed to embodiments of the present disclosure, other and further embodiments of the disclosure may be devised without departing from the basic scope thereof, and the scope thereof is determined by the claims that follow.

What is claimed is:

1. A method of forming a microelectronic device, the method comprising:
pre-treating a metal surface formed on a substrate;
depositing a self-assembled monolayer (SAM) selectively on the metal surface against a dielectric surface formed on the substrate;
depositing a barrier layer selectively on the dielectric surface against the SAM; and
removing the SAM.

2. The method of claim 1, wherein the metal surface comprises copper (Cu), cobalt (Co), tungsten (W), molybdenum (Mo), ruthenium (Ru), titanium nitride (TiN), or any combination thereof.

3. The method of claim 1, wherein pre-treating the metal surface comprises soaking the substrate in nitrogen containing molecules or gas.

4. The method of claim 1, wherein depositing the SAM comprises soaking the substrate in blocking molecules.

5. The method of claim 4, wherein the blocking molecules comprise alkyne molecules.

6. The method of claim 1, wherein the barrier layer comprises one or more of tantalum nitride (TaN), titanium nitride (TiN), silicon nitride (Si₃N₄), titanium silicon nitride (TiSiN), tantalum silicon nitride (TaSiN), or silicon oxynitride (SiON).

7. The method of claim 1, further comprising:
selectively depositing a metal liner on the barrier layer.

8. The method of claim 7, wherein the metal liner comprises ruthenium (Ru), cobalt (Co), molybdenum (Mo), tantalum (Ta), or any combination thereof.

9. A method of forming a microelectronic device, the method comprising:
pre-cleaning and pre-treating a top surface of a first metal layer exposed within a feature opening extending through a dielectric layer that is formed on the first metal layer;
depositing a blocking layer selectively on the top surface of the first metal layer exposed within the feature opening against sidewalls of the feature opening;
depositing a barrier layer selectively on the sidewalls of the feature opening against the blocking layer;
removing the blocking layer; and
filling the feature opening with a second metal layer.

10. The method of claim 9, wherein the first metal layer comprises copper (Cu), cobalt (Co), tungsten (W), molybdenum (Mo), ruthenium (Ru), titanium nitride (TiN), or any combination thereof.

11. The method of claim 9, wherein pre-treating the top surface of the first metal layer comprises soaking the top surface of the first metal layer in nitrogen containing molecules or gas.

12. The method of claim 9, wherein depositing the blocking layer comprises soaking the top surface of the first metal layer in blocking molecules.

13. The method of claim 12, wherein the blocking molecules comprise alkyne molecules.

14. The method of claim 9, wherein the barrier layer comprises one or more of tantalum nitride (TaN), titanium nitride (TiN), silicon nitride (Si₃N₄), titanium silicon nitride (TiSiN), tantalum silicon nitride (TaSiN), or silicon oxynitride (SiON).

15. The method of claim 9, further comprising:
prior to filling the feature opening, selectively depositing a metal liner on the barrier layer.

16. The method of claim 15, wherein the metal liner comprises ruthenium (Ru), cobalt (Co), molybdenum (Mo), tantalum (Ta), or any combination thereof.

17. The method of claim 9, wherein the second metal layer comprises copper (Cu) or cobalt (Co).

18. A method of removal of self-assembled monolayer (SAM) from a metal surface, the method comprising:
soaking the metal surface in ammonia (NH₃) gas supplied at a flow rate of between 500 sccm and 3000 sccm, for a time duration of between 10 seconds and 60 seconds;
subsequent to soaking the metal surface, depositing a self-assembled monolayer (SAM) on the metal surface; and
removing the SAM from the metal surface, wherein carbon (C) residue on the metal surface after the removing the SAM is reduced as compared to a SAM removal without the soaking the metal surface.

19. The method of claim 18, wherein the metal surface comprises copper (Cu), cobalt (Co), tungsten (W), molybdenum (Mo), ruthenium (Ru), titanium nitride (TiN), or any combination thereof.

20. The method of claim 18, wherein depositing the SAM comprises soaking the metal surface in blocking molecules comprising alkyne molecules.
